(19) United States
(12) Patent Application Publication
(10) Pub. No.: US 2017/0326162 A1
(43) Pub. Date: Nov. 16, 2017

(54) Demineralized Bone Matrix Material having Sphingosine-1-Phosphate or Ceramide-1-Phosphate

(71) Applicant: DePuy Synthes Products, Inc., Raynham, MA (US)

(72) Inventor: Thomas M. DiMauro, Southboro, MA (US)

(73) Assignee: DePuy Synthes Products, Inc., Raynham, MA (US)

(21) Appl. No.: 15/586,630

(22) Filed: May 4, 2017

Related U.S. Application Data
(60) Provisional application No. 62/337,047, filed on May 16, 2016.

(51) Int. Cl. A61K 31/661; A61K 47/36; A61K 35/32

(52) U.S. Cl. CPC A61K 31/661; A61K 35/32; A61K 47/36

(57) ABSTRACT

A new composition has been developed that incorporates S1P into a conventional demineralized bone matrix material. Once the device is implanted into the spine, the S1P will elute out of the device, thereby setting up a concentration gradient in the vicinity of the device. This gradient will cause stem cells to preferentially migrate to the device.

### CONTINUING DATA

This application claims priority from co-pending application U.S. Ser. No. 62/337,047, filed May 16, 2016 (DiMauro), entitled “Demineralized Bone Matrix Material having Sphingosine-1-Phosphate or Ceramide-1-Phosphate”, Docket No. DSP5242USPSP, the specification of which is incorporated by reference in its entirety.

### BACKGROUND OF THE INVENTION

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contained sulfated functional groups that retain water, thereby providing the nucleus pulposus within its cushioning qualities. These nucleus pulposus cells may also secrete small amounts of cytokines as well as matrix metalloproteinases (“MMPs”). These cytokines and MMPs help regulate the metabolism of the nucleus pulposus cells.

In some instances of disc degeneration disease (DDD), gradual degeneration of the intervertebral disc is caused by mechanical instabilities in other portions of the spine. In these instances, increased loads and pressures on the nucleus pulposus cause the cells to emit larger than normal amounts of the above-mentioned cytokines. In other instances of DDD, genetic factors, such as programmed cell death, or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins.

As DDD progresses, the toxic levels of the cytokines present in the nucleus pulposus begin to degrade the extracellular matrix (in particular, the MMPs (under mediation by the cytokines) begin cleaving the water-retaining portions of the proteoglycans, thereby reducing its water-retaining capabilities). This degradation leads to a less flexible nucleus pulposus, and so changes the load pattern within the disc, thereby possibly causing delamination of the annulus fibrosus. These changes cause more mechanical instability, thereby causing the cells to emit even more cytokines, thereby upregulating MMPs. As this destructive cascade continues and DDD further progresses, the disc begins to bulge (“a herniated disc”), and then ultimately ruptures, causing the nucleus pulposus to contact the spinal cord and produce pain.

Patients suffering from DDD often conclude that a highly invasive surgery called a fusion is necessary for their condition. In a fusion, the degenerating disc is removed and replaced with a synthetic cage containing bone graft. The opposed vertebral bodies above and below the cage fuse to each other over the space of a year and stabilize the region, often producing pain relief. However, fusion success rates, as defined by pain relief, have remained at about 50-60% for the last 15 years.

### (DBM)

When a fusion is selected for the patient, the surgeon often uses a fusion cage filled with a fusion-facilitating composition such as demineralized bone matrix (DBM). As its name implies, DBM is the product of removing the mineral content from bone, thereby leaving collagen and a host of growth factors that are conductive to bone growth. It is believed the DBM helps the patient attain a successful fusion by facilitating bone growth across the disc space. The growth factors in DBM include TGF-β and PDGF.

### (Stem Cells)

Stem cells comprise about 1.1% of all the nucleated cells within bone marrow. Korbling, Blood, 15 Nov. 2001, 98, 10, 2900-2908. Two types of stem cells are present in this marrow—hematopoietic stem cells (HSCs) and mesenchymal stem cells (MSCs). Whereas HSCs have the ability to transform into blood-based cells, the MSCs have the ability to transform into both bone cells and cartilage cells. Therefore, there is great interest in exploiting MSCs for their potential use in intervertebral fusion and intervertebral disc repair.

### SUMMARY OF THE INVENTION

It has been reported that sphingosine-1-phosphate (S1P) is a powerful chemotactic agent for mesenchymal stem cells.

Accordingly, a new composition has been developed that incorporates S1P into a conventional demineralized bone matrix material. Once the device is implanted into the spine, the S1P will elute out of the device, thereby setting up a concentration gradient in the vicinity of the device. This gradient will cause stem cells to preferentially migrate to the device.

It is believed that the novel composition will provide superior results in fusion situations. The S1P component will cause mesenchymal stem cells (MSCs) present in the bone marrow component of adjacent vertebral bodies to preferentially migrate to the fusion cage. Once inside the implanted fusion cage, the MSCs will be susceptible to osteogenic signaling and thereby transform into bone cells. These bone cells will increase the likelihood of a successful fusion, thereby increasing the chances for the surgery to be a pain-relieving event.

Therefore, in accordance with the present invention, there is provided (claim 1).

### DETAILED DESCRIPTION OF THE INVENTION

As a potential replacement for fusions, autologous stem cell therapy has been proposed as a potential solution for DDD. The stem cells can be obtained from the patient's bone marrow or adipose tissue, concentrated, and then injected into the disc. Once in the disc, these stem cells may either emit trophic factors that support the native cells of the disc and/or receive signals that enable them to differentiate into chondrocytes that form nucleus pulposus cells. In is hoped that these new nucleus pulposus cells can help rehydrate the disc and restore disc height, thereby alleviating pain.

US Patent Publication US 2014-0335055 (Pettine) discloses a needle-based autologous cell therapy concentrate made by mixing a pre-determined amount of a processed bone marrow cellular matrix comprising centrifuge-concentrated stem cells with a substantially equal amount (by volume) of a pre-mixture comprising substantial equal volumes of a) an anticoagulant solution (such as ADCA), b) 50% dextrose solution, and c) phosphate buffered saline. Pettine's premixture is believed to augment the viability of the stem cells once they are injected into the harsh environment of the disc. For the sake of clarity, the Pettine process can be summarized as follows:

Pettine discloses injecting the autologous cell therapy concentrate into a degenerating disc of 26 DDD patients who were previously determined to be surgery candidates and reports promising results.

### (Disc Repair)

It is further believed that the novel composition also has utility in regenerating a degenerated intervertebral disc. Pettine, Stem Cells, 2015, 33, 146-156 has reported that injecting MSCs from centrifuged bone marrow aspirate into a degenerating disc results in long lasting pain relief for a substantial majority of the DDD patients undergoing the procedure. However, Pettine also reports that a significant fraction of the patients having an inadequate concentration of MSCs in their centrifuged BMA (i.e., less than 2000 CFU-F/ml) did not experience significant pain relief.

Therefore, it is believed there would be utility in increasing the MSC concentration in the Pettine injectate, and that exposing the BMA to a composition loaded with chemotactic agents would preferentially draw the MSCs from the BMA into the composition.

It is believed that the novel composition possesses a number of features that would provide utility when the composition is used as an injectable for regenerating a degenerated disc. In short, the composition possesses a number of chemotactic agents that attract MSCs. These chemotactic agents can be used to preferentially draw the MSCs from the BMA into the composition, thereby seeding the composition with MSCs that can become nucleus pulposus cells.

### (Components)

(DBM)

First, the composition comprises DBM. As discussed above, DBM possesses a substantial amount of growth factors. At least the TGF-β and PDGF components in DBM are chemotactic agents for MSCs. In addition, the PDGF has been reported to increase the expression of CD44 in MSCs. Zhu, Stem Cells, 2006, 24, 928-935. This is important because CD44 is the receptor for hyaluronic acid and so plays a role in MSC chemotaxis. See Zhu, supra, and Bian, Kidney Blood Press. Res., 2013, 38, 1, 11-20. Accordingly, the PDGF component of the DBM appears to work synergistically with the HA component of the composition.

(Hyaluronic Acid)

Second, the composition further comprises hyaluronic acid. HA has been reported to be a chemotactic agent for mesenchymal stem cells. Bian, Kidney Blood Press. Res., 2013, 38, 1, 11-20; and Zhu, Stem Cells, 2006, 24, 928-935. Bian reported that HA concentrations of between 100 and 300 ug/ml induced significant MSC migration.

In some embodiments, the hyaluronate component of the hyaluronate-based carrier has a molecular weight ranging from five hundred thousand to three million Daltons. In some embodiments, the hyaluronate-based component of the carrier comprises from about 1.0% to about 10.0% by weight (wt %) of the carrier, preferably between about 2 wt % and about 5 wt %, with the remainder substantially comprising water.

Moreover, it has been reported in the literature that the DBM and HA components are conducive to growing cartilage. In particular, investigators have reported that DBX, a composition comprising 31 wt % DBM in a 4% HA carrier, was suitably used as a carrier for BMP-2 for cartilage repair in rabbit and goat models. Frenkel, 51^{st} Annual Mtg. ORS, Poster No. 1796, 2005.

(S1P/C1P)

Lastly, the composition further comprises an agent selected from the group consisting of sphingosine-1-phosphate (SIP) and ceramide-1-phosphate (C1P). S1P has been reported to be a chemotactic agent for mesenchymal stem cells. Quint, J. Biol. Chem., 288, 8, 5398-5406, 2013, and Kong, Mediators of Inflammation, 2014, ID 565369. Kong reported that S1P concentrations between 0.1 nM and 50 nM induced migration of MSCs, with the greatest migrations being induced by concentrations between 1 nM and 50 nM. See Kong at FIG. 2. Therefore, in some embodiments of the present invention, the composition has a S1P concentration of between 0.1 nM and 50 nM, preferably between 1 nM and 50 nM.

C1P has been reported to be a chemotactic agent for mesenchymal stem cells. Kim, Stem Cells, 2013 March; 31(3), 500-510. Kim reported chemotactic activity for solution having 0.1 and 1 uM C1P. Therefore, in some embodiments, the composition contains between 0.01 and 10 uM C1P, preferably between 0.1 and 1 uM C1P.

### (All are Endogenous)

Each of these chemotactic agents is present in large amounts in the human body. For example, hyaluronic acid is present in extracellular matrix in the human body in an amount of at least 2.5 g/L. See Sigma-Aldrich, citing Chemistry and Biology of Hyaluronan H. G. Garg and C. A. Hales (editors) 2004 Elsevier; Chapter 4: Biodegradation of Hyaluronan (G. Lepperdinger, C. Fehrer, S. Reitinger). According to Hammad, Journal of Lipids, Vol. 2012 (2012), Article ID 180705, S1P is found in red blood cells in plasma at relatively high concentrations (>200 nM in plasma). According to Ratajczak, Expert Opin. Ther. Targets, 2014, Jan. 18, 1, 95-107, the concentration of C1P in peripheral blood (PB) is relatively high, ˜0.5-1 μM, and is much lower in interstitial fluids. Because these endogenous agents appear in high concentrations in the human body, the safety of the composition appears to be evident.

### (Component Fractions)

In preferred compositions, the demineralized powder comprises about 15% to about 40% of the weight of the composition, the carrier comprises between about 50 wt % and 80 wt % of the composition, and the S1P/C1P component consists essentially of the remainder.

### (Enrichment)

Furthermore, not only does the novel composition increase the MSC content in the injectable, it also produces enrichment of the MSC component with the total number of cells. It is believed that these chemotactic agents will draw only stem cells (HSCs and MSCs) into the composition. Because these stem cells comprise only about 1.1% of the total nucleated cells in BMA, it is believed the novel composition will have a stem cell concentration that is about 100-fold enriched for stem cells over the conventional BMA. This is important because there is substantial concern for the overall concentration of cells in the injectate. In particular, there is concern that, due to endplate sclerosis, nutrition within the disc is already scarce and that injecting too many cells into a degenerating disc will cause fierce competition between the cells for the scarce nutrition, leading to an overall unfavorable result. In this respect, the stem cells in a composition that is 100-fold enriched for stem cells (over conventional BMA) will have a much easier time competing for the scarce nutrition, and so will more likely survive and thrive in the disc, thereby leading to disc repair.

### (How to Make)

In some embodiments, the novel composition is made by simply mixing S1P (available from Sigma-Aldrich, St. Louis, Mo., USA)) with DBX, a demineralized bone matrix material having a hyaluronate carrier (available from DePuy Synthes Spine, Raynham, Mass.). In some embodiments, the composition is made by simply mixing S1P (available from Sigma-Aldrich, St. Louis, Mo., USA)) with the DBM-HA composition DBX disclosed in U.S. Pat. No. 6,030,635, the specification of which is incorporated by reference in its entirety.

### (Method)

In accordance with the present invention, there is provided a method comprising:
a) aspirating bone marrow from the patient,
b) centrifuging the bone marrow aspirate to produce a bone marrow concentrate,
c) combining the bone marrow concentrate with the composition of the present invention ex vivo to allow selective stem cell migration from the concentrate into the composition, and
d) injecting the stem cell-containing composition into the degenerating disc of the patient.

### (In-Situ Seeding)

Whereas the previous paragraph discloses the ex vivo seeding the composition of the present invention with MSCs from bone marrow aspirate, it is also envisioned that the seeding can take place within the body. That is, the composition can be placed within the bone marrow of a bone (for example, the iliac crest) and allowed to remain there for a sufficient time for MSCs from the marrow to migrate into the composition. After a period of time, the MSC-laden composition can be retrieved from the marrow (through a needle) and then placed into the patient's spine. This method has the advantage of maximizing the time allowed for migration and seeding, and therefore has the greatest chance for having a high MSC concentration in the injectate.

Therefore, in accordance with another aspect of the present invention, there is provided a method comprising:

a) selecting a composition comprising:
i) demineralized bone powder,
ii) a hyaluronate-based carrier, and
iii) a chemotactic agent selected from the group consisting of sphingosine-1-phosphate and ceramide 1-phosphate,

b) injecting the composition into bone marrow residing in the patient,

c) allowing MSCs present in the bone marrow to infiltrate the composition,

d) withdrawing the MSC-laden composition from the bone marrow, and

e) injecting the MSC-laden composition into the spine of a patient.

### (Adipose)

It is further believed that the novel composition may also be used to selectively extract stem cells from adipose tissue as well. If adipose tissue is selected, the adipose tissue is first preferably enzymatic digested (for example with collagenase) to remove the cells from the extracellular matrix, and then the free cells are centrifuged to stratify the free cells so that they are amenable to concentration.

In one preferred embodiment, adipose tissue (2000 ml) recovered from liposuction is washed in PBS and digested with collagenase type I. After centrifugation at 500×g at room temperature, mature adipocytes in the supernatant are discarded and the pellet is identified as the SVF. The cells may be seeded in a culture flask, at a density of 3×10⁵ cells/cm2, to establish a primary culture.

Novel compositions derived from adipose tissue are especially attractive because the native adipose tissue will have substantially no hemopoietic cell (that are also amenable to migration when exposed to the chemotactic agent), and so the selectivity accomplished by the chemotactic agent be greatly increased because substantially only mesenchymal stem cells undergo migration.

### (Cinnamtannin B-1)

In some embodiments, cinnamtannin B-1 may be used as a chemotactic agent. Cinnamtannin B-1 is a phytochemical present in cinnamon. Investigators have reported that Cinnamtannin B-1 induces chemotaxis of MSCs both in vitro and in vivo. Furumoto, Phytomedicine, 21 (2014) 247-253 and Fujita, PLOS ONE, 10(12):e0144166 DOI:10.1371. Furumoto reported that Cinnamtannin B-1 concentrations between 0.8 ug/ml and 20 ug/ml induced the greatest migration of MSCs. Therefore, in some embodiments of the present invention, the composition has a Cinnamtannin B-1 concentration of between 0.8 ug/ml and 20 ug/ml.

I claim:

1. A sterile malleable composition for application to a spinal site to promote new tissue growth at the site, comprising:
i) a mixture of demineralized bone powder,
ii) a hyaluronate-based carrier, and
iii) a chemotactic agent selected from the group consisting of sphingosine-1-phosphate and ceramide 1-phosphate.

2. The composition of claim 1 wherein the powder has a median particle size ranging from about 100 to about 850 microns.

3. The composition of claim 1 wherein powder comprises about 15% to about 40% of the weight of the composition, and the carrier comprises between about 50 wt % and 80 wt % of the composition.

4. The composition of claim 1 wherein the hyaluronate component of the hyaluronate-based carrier has a molecular weight ranging from five hundred thousand to three million Daltons.

5. The composition of claim 1 wherein a hyaluronate-based component of the carrier comprises from about 1.0% to about 10.0% by weight of the carrier solution.
